(19) United States
(12) Patent Application Publication
(10) Pub. No.: US 2012/0253105 A1
(43) Pub. Date: Oct. 4, 2012

(54) METHOD OF ANCHORING AN ACOUSTIC ELEMENT IN A BONE OF THE CRANIOMAXILLOFACIAL REGION AND ACOUSTIC ELEMENT

(75) Inventor: Jorg Mayer, Niederlenz (CH)

(73) Assignee: WOODWELDING AG, Zug (CH)

(21) Appl. No.: 13/502,622

(22) PCT Filed: Oct. 20, 2010
(86) PCT No.: PCT/CH2010/000266

§ 371 Date: Jun. 21, 2012

Related U.S. Application Data
(60) Provisional application No. 61/253,646, filed on Oct. 21, 2009.

(51) Int. Cl. A61F 11/04; H04R 25/02; H04R 25/00

(52) U.S. Cl. 600/25; 607/57; 381/328

(57) ABSTRACT

An acoustic element is implanted in a human bone of the craniomaxillofacial region with the aid of at least one anchor element including a material having thermoplastic properties, wherein for implantation of the acoustic element an opening is provided in the bone, the anchor element is positioned in the bone and energy is transmitted into the material having thermoplastic properties for at least partly liquefying the material and making it penetrate the bone tissue in the opening and on re-solidification to constitute a positive fit connection between the anchor element and the bone tissue. Depending on the design of the anchor element and/or on the relative dimensions of the opening and the anchor element it is possible to achieve an anchorage which either transmits sound between the acoustic element and the bone tissue or does not do so.

### FIELD OF THE INVENTION

The invention belongs to the field of otology and concerns a method for anchoring an acoustic element (element having an acoustic function), in particular an implantable hearing aid or an implantable component of a hearing aid, in a bone of the craniomaxillofacial region, in particular in a human bone of the craniomaxillofacial region. The invention further concerns an acoustic element which is suitable to be anchored in a bone with the aid of the method according to the invention.

### BACKGROUND OF THE INVENTION

The human hearing facility is mainly based on the ear which receives sound with the aid of the pinna and transmits it through the outer auditory canal to the tympanic membrane, the auditory ossicles of the tympanic cavity and the oval window (auditory chain) to the cochlea where nerve ends sense the mechanical sound and transmit corresponding signals to the brain. Another important aspect of human hearing is the direct transmission of sound from the skull functioning as a resonator, to the cochlea, i.e. the transmission of sound not through the auditory chain but through bones of the craniomaxillofacial region. For this reason human hearing can be supported by coupling desired sound into bones of the craniomaxillofacial region but obviously it may be greatly disturbed by coupling undesired sound into such bones.

There are many types of hearing aids on the market. Regarding their functioning principle, there are substantially three categories of hearing aids: hearing aids acting mechanically on the ear, e.g. by delivering sound to the auditory canal, to the tympanic cavity or to the cochlea; hearing aids acting electrically on the auditory nerves, e.g. hearing aids comprising a cochlear implant (electrode implanted in the cochlea); and hearing aids acting mechanically on the skull (so called bone anchored hearing aids), i.e. hearing aids which transmit sound to cranial bones, which bones then further transmit the sound to the cochlea.

The main components of a hearing aid are an input transducer (microphone), a sound processor with at least an amplifying function and an output transducer (loudspeaker or actuator in hearing aid acting mechanically or electrode in hearing aid acting electrically).

For all three named hearing aid categories there are proposals according to which at least specific components of the hearing aid are implanted, in particular anchored in bones of the craniomaxillofacial region. A few examples of such implantable components are, in addition to the cochlear implant, the following:

Output tranducers of hearing aids acting mechanically on the skull or complete such hearing aids are releaseably fixed to a transdermal anchor element which is anchored in a cranial bone (as e.g. disclosed in U.S. Pat. No. 4,498,461 and WO-2005/037153).

Output transducers of hearing aids acting mechanically on the cochlea are anchored in the promontory of the otic capsule bone (as e.g. disclosed in U.S. Pat. No. 5,951,601).

Output transducers and possibly also input transducers of middle ear hearing aids (acting mechanically on the ear) are implanted in the middle ear and anchored in the mastoid bone (as e.g. disclosed in U.S. Pat. No. 6,001,129).

Sound processing components are implanted subcoutaneously behind the ear (as e.g. disclosed in U.S. Pat. No. 5,951,601).

Input transducers (internal receiver and stimulator) cooperating with a cochlear implant are secured to bone and inductively receive signals from outer components which signals they transmit to the cochlear electrode via an internal cable.

According to the state of the art, anchorage of hearing aids or hearing aid components in bones of the craniomaxillofacial region is effected by bone screws which advantageously are equipped for osseointegration, as e.g. bone screws of titanium comprising in a per se known manner a surface structure or surface coating which is capable of enhancing osseointegration. Such screw anchorage has sound transmitting properties which cannot be exactly anticipated and tend to change over time. Immediately after implantation sound transmission will be high, will then decrease due to bone relaxation around the screw and then increase again with progression of osseointegration, wherein osseointegration may be hampered by loosening of the bone screw due to loads put on the screw and also due to sonic vibration. Only a fully osseointegrated bone screw is able to transmit sound as required e.g. by a so called bone anchored hearing aid. Due to the named difficulties separate damping means (e.g. elastomeric damping means as disclosed in U.S. Pat. No. 5,951,601) are provided where sound transmission through the anchorage is not desired. Furthermore, it is highly probable that the same difficulties are the reason for failures on implantation of transdermal anchor elements for so called bone anchored hearing aids, which failures prompt surgeons to enhance the probability for one successful anchor element by straight away implanting a plurality of such anchor elements and wait for six to eight months (for complete osseointegration) before the hearing aid or part thereof is coupled to the anchor element.

The above shows that the screw anchorage of an acoustic element cannot fully satisfy many conditions regarding sound transmission between an acoustic element and the bone in which the screw is anchored, which conditions differ largely between different acoustic elements dependent on the acoustic function of the element.

### BRIEF SUMMARY OF THE INVENTION

It is the object of the invention to provide a method for anchoring an acoustic element (element having an acoustic function) in a bone of the craniomaxillofacial region, wherein the acoustic properties of this anchorage are to satisfy largely different requirements of differing acoustic elements and are to be easily adaptable to such differing requirements. This means in particular that the anchorage achieved by the method according to the invention either transmits sound from the acoustic element to the bone (or in the opposite direction) with minimal loss and consistently as from the moment of implantation or does not do so (i.e. with maximal loss) without the need of separate damping means. It is a further object of the invention to create an acoustic element which is suitable for being anchored in a bone of the craniomaxillofacial region using the method according to the invention.

These objects are achieved by the method and the acoustic element as defined in the appended claims.

The bases of the method according to the invention are anchoring techniques which work with the aid of an anchor element and provision of an opening in the bone for implanting the anchor element, wherein the anchor element comprises a material with thermoplastic properties, i.e. being liquefiable by thermal energy, which material is arranged on the anchor element such that it can be liquefied in situ and, in its liquefied state, be made to penetrate natural or specifically provided cavities, pores or other suitable structures of bone tissue in the opening provided for the anchor element, where on re-solidification it forms a positive fit connection between the anchor element and the bone tissue. The energy needed for the in situ liquefaction is coupled during implantation into the anchor element as vibrational energy, rotational energy, electromagnetic radiation (laser light in the visible or infrared frequency range) or electric energy, to be transformed into thermal energy where liquefaction is desired.

For achieving differing sound transmitting properties of the anchorage the arrangement of the material with thermoplastic properties on the anchor element is adapted and possibly differing such materials are chosen. The anchoring method however remains the same.

If the method/element is in accordance with the first object of the invention (i.e. optimal coupling of sound, for example over its full audible spectrum into the bone or vice versa, for example by an osseo-integrative, bony connection of the actuator in the skull bone)—i.e. if the anchor element is to transmit sound between the acoustic element and the bone—then the anchor element comprises a transmitter core made of a material which is able to transmit sound with minimal damping (e.g. metal such as e.g. titanium or a titanium alloy or a ceramic material such as e.g. zirconium oxide). For being capable of functioning as sound transmitting means, the transmitter core firstly needs to be rigidly and directly (no damping means therebetween) attached or attachable to other parts of the acoustic element, and secondly in the implanted state of the anchor elements it needs to be in intimate contact with the bone tissue or the opening provided for the anchor element in some core surface regions (no interface of liquefiable material between such core surface regions and the bone tissue), wherein at least these core surfaces need to be equipped for furthering osseointegration in a per se known manner. The positive fit connection to be established with the aid of the material having thermoplastic properties is to occupy other core surface regions. Immediately after implantation, the positive fit connection between the anchor element and the bone tissue keeps the transmitter core in intimate contact with the bone tissue and ensures good sound transmission. As this positive fit connection is not impaired by bone relaxation and not prone to be loosened by loads put on the anchor element or by transmitted sound, sound transmission is not deteriorating after implantation. Preferably the material having thermoplastic properties is bioresorbable and the whole surface of the transmitter core is equipped for enhancing osseointegration such that the named positive fit connection is gradually replaced by osseointegration. If the bone in which the anchor element is to be anchored does not have the mechanical properties which are necessary for a secure anchorage, the material having thermoplastic properties is chosen to be non-bioresorbable, wherein the liquefied material penetrated into the bone tissue not only forms the named positive fit connection, but also strengthens the bone tissue in the location of the anchorage.

For allowing intimate contact between the transmitter core of the anchor element and the bone tissue, the transmitter core is to fit tightly into the opening provided for the anchoring at least in one direction, wherein at least in the regions of such tight fit, the surfaces of the anchor element (transmitter core and material having thermoplastic properties) are preferably even to concave and the material having thermoplastic properties either fills recesses in the core surface or is arranged or arrangeable in a core constituting a hollow and perforated or fenestrated sleeve or tube.

The above named anchor element, which after implantation will transmit sound virtually loss-free and in an unchanging manner from the acoustic element to the bone in which it is anchored or possibly in the opposite direction, is e.g. highly suitable for anchoring a so called bone anchored hearing aid or component thereof to a human skull bone, wherein the anchor element can bear loads and the hearing aid or component thereof can be used immediately after implantation and therefore the hearing aid or component thereof may be rigidly and permanently coupled to the anchor element and be implanted together with the latter.

If the method/element is in accordance with the second object of the invention (i.e. maximal, controlled damping of sound; minimizing the coupling of sound into the skull or, for example for a microphone, from the skull for example over its full audible spectrum—i.e. if the anchor element is not to transmit sound, neither from the acoustic element to the bone in which it is to be anchored, nor in the opposite direction (sonic element and bone are to be sonically uncoupled),—then the anchor element itself is designed as a damping element (damping function integrated in the anchor element), i.e. the liquefiable material constitutes the whole anchor element or at least after implantation constitutes a full interface between a core (which may be made of a sound transmitting material, i.e. be quite similar to the above mentioned transmitter core) and the bone tissue in the opening provided for the anchorage or distances core surfaces not covered with it from this bone tissue. Furthermore, the material having thermoplastic properties is preferably chosen to have elastic properties suitable for the damping function. It is possible also to equip the anchor element with two materials having thermoplastic properties, wherein the damping material to be liquefied (for example with modulus of elasticity <0.5 GPa) being enclosed in a hollow pin made of the material to be at least partly liquefied on implantation (for example with modulus of elasticity >0.5 GPa). Depending on whether the anchorage is to be a permanent feature or a temporary one, the material having thermoplastic properties and the material of the core are chosen to both be either bioresorbable or non-bioresorbable. An implanted anchor element of which the material of the positive fit connection is bioresorbed and a rest of the implant (not resorbed yet or not resorbable), in particular a sound transmitting core is to be prevented as it may cause undesired sound transmission.

The anchor element with integrated damping function is e.g. suitable for anchoring a microphone (input transducer) to a cranial bone, an input and/or output transducer of a middle ear hearing aid in the mastoid bone or in the outer auditory canal, or an output transducer in the otic capsule bone.

Apart from the above discussed easy adaptability of the acoustic element or its anchor element respectively for differing requirements regarding sound transmission between the acoustic element and the bone in which it is anchored, the method according to the invention is furthermore particularly suitable for the present purpose, because usage of the method makes it possible to produce mechanically stable anchorages even if an opening in the bone provided for the anchor element has a very small depth of a few millimeters only (thickness of cranial bone: 4 mm, anchorage possible in a blind bore), because it allows to sealingly close this opening which is particularly important if the concerned bone separates the outer world from the highly infection sensitive inner ear or brain, and because it does not necessitate a circular cross section of the opening as a screw does.

The anchor element may be part of the acoustic element, wherein the acoustic element is e.g. a hearing aid or hearing aid component.

According to a first option, the at least one anchor element may constitute an integral part of the acoustic element and the acoustic element together with the anchor element is implanted in a one-step procedure.

According to a second option, the at least one anchor element, before implantation, constitutes a separate item, and the implantation is a two- or multi-step procedure, wherein e.g. the anchor element is implanted first and the acoustic element is then coupled to the implanted anchor element.

According to yet a further option, the at least one anchor element before implantation constitutes a separate element and is implanted after the acoustic element is put into place. It is then connected to both, the bone tissue and the acoustic element. This may be achieved for example by pushing the at least one anchor element between the bone tissue and the acoustic device. To this end, according to a first possibility the acoustic element may comprise liquefiable material, too, and by the pushing the anchor element between the tissue and the acoustic element while energy impinges, a welding connection is created between the acoustic element and the anchor element—in addition to the anchoring of the anchor element in the bone tissue. According to a second possibility, one of the anchor element and the acoustic element comprises a structure which is suitable to form a positive-fit connection, and liquefied material of the other one of the anchor element and the acoustic element interpenetrates the structure to form, after re-solidifying, a positive-fit connection therewith, in addition to the anchoring in the bone tissue.

In accordance with this option, therefore, attaching of the anchor element to the acoustic element is carried out simultaneously with anchoring.

By the technique of inserting, under impingement of energy, an anchor element after the acoustic element is inserted, may have the further advantage that cavities temporarily created for implantation or other spacings may be filled by the liquefied material.

The above named anchoring techniques working with the aid of in situ liquefaction of a material having thermoplastic properties, on which techniques the method according to the invention is based as well as implants or anchor elements respectively being suitable for the implantation techniques are disclosed e.g. in the publications U.S. Pat. No. 7,335,205, U.S. Pat. No. 7,008,226, US 2006/0105295, and US-2008/109080, WO 2009/055 952, and WO 2009/132 472, as well as in U.S. patent application Ser. No. 61/388,243. A technique that involves pushing the at least one anchoring element between the bone tissue and the acoustic element subsequent to placing the acoustic element in place is disclosed in WO 2008/034 276, especially referring to FIGS. 23-34. The entire disclosure of all the named publications and applications is incorporated herein by reference.

As already mentioned further above, the principle of the named implantation techniques is the in situ liquefaction of a material having thermoplastic properties such that in its liquefied state it has a viscosity which enables it to penetrate into natural or beforehand provided pores, cavities or other structures of the bone tissue, and wherein an only relatively small amount of the material is liquefied such that no unacceptable thermal load is put on the tissue. Suitable liquefaction connected with an acceptable thermal loading of the tissue is achievable by using materials with thermoplastic properties having a melting temperature of up to about 350° C. and by providing such material e.g. on surfaces of the anchor element, which on implantation are pressed against the bone tissue, preferably by introducing the anchor element in a bone opening which is slightly smaller than the anchor element or by expanding the anchor element in a bone opening which originally is slightly larger than the anchor element (expansion e.g. by mechanically compressing or buckling the anchor element). For some embodiments, it is advantageous if the modulus of elasticity of the material with thermoplastic properties is at least 0.5 GPa. During implantation, the anchor element is subjected to vibration of a frequency preferably in the range of between 2 and 200 kHz (preferably ultrasonic vibration), especially above 25 kHz to be above the auditory threshold, especially between 25 kHz and 35 kHz by applying e.g. the sonotrode of an ultrasonic device to the anchor element or to another portion of the implantable acoustic element. In embodiments in which the thermoplastic material has a relatively high modulus of elasticity, the thermoplastic material transmits the ultrasonic vibration with such little damping that inner liquefaction and thus destabilization of the fusion device does not occur, i.e. liquefaction occurs only where the liquefiable material is in contact with the bone tissue and is therewith easily controllable and can be kept to a minimum. In other embodiments, for example where a filling effect has to be achieved or where a thermoplastic element is consumed to a large extent to be pressed out of a not liquefied sheath, the modulus of elasticity may be chosen to be relatively lower.

Instead of providing the liquefiable material on the surface of the implant (disclosed e.g. in U.S. Pat. No. 7,335,205 or U.S. Pat. No. 7,008,226), it is possible also to provide the liquefiable material in a perforated sheath and to liquefy it within the sheath and press it through the sheath perforation to the surface of the fusion device and into the pores or cavities of the bone tissue (disclosed e.g. in U.S. Pat. No. 7,335,205, U.S. Pat. No. 7,008,226 and U.S. provisional application 61/0495879) and/or it is possible to liquefy the liquefiable material between two implant parts of which one is vibrated and the other one serves as counter element, the interface between the two implant parts being positioned as near as possible to the bone tissue (as disclosed in the U.S. provisional applications 60/983,791 and 61/049587).

Instead of using vibrational energy for creating the local thermal energy needed for the liquefaction of the material with thermoplastic properties, it is possible also to exploit other energy types, in particular rotational energy turned into friction heat in substantially the same manner as the vibrational energy, or electromagnetic radiation (in particular laser light in the visible or infrared frequency range), which radiation is preferably guided through the material with thermoplastic properties and locally absorbed by an absorber being contained in the material with thermoplastic properties or being arranged adjacent to this material.

Suitable liquefiable materials to be used for the anchor element are thermoplastic polymers. A first group is resorbable polymers such as polymers based on lactic and/or glycolic acid (PLA, PLLA, PGA, PLGA etc.) or polyhydroxy alkanoates (PHA), polycaprolactone (PCL), polysaccharides, polydioxanes (PD) polyanhydrides, polypeptides or corresponding copolymers or composite materials containing the named polymers as a component. Examples of suited thermoplastic material include any one of the polylactide products LR708 (amorphous Poly-L-DL lactide 70/30), L209 or L210S by Bohringer Ingelheim.

A second group is non-resorbable polymers such as polyolefines (e.g. polyethylene), polyacrylates, polymetacrylates, polycarbonates, polyamides, polyester, polyurethanes, polysulfones, polyarylketones, polyimides, polyphenylsulfides or liquid crystal polymers LCPs, polyacetales, halogenated polymers, in particular halogenated polyolefines, polyphenylensulfides, polysulfones, polyethers, polypropylene (PP) or corresponding copolymers or blended polymers or composite materials containing the named polymers as a component.

Specific embodiments of degradable materials are Polylactides like LR706 PLDLLA 70/30, R208 PLDLA 50/50, L210S, and PLLA 100% L, all by Böhringer. A list of suitable degradable polymer materials can also be found in: Erich Wintermantel und Suk-Woo Haa, “Medizinaltechnik mit biokompatiblen Materialien und Verfahren”, 3. Auflage, Springer, Berlin 2002 (in the following referred to as “Wintermantel”), page 200; for information on PGA and PLA see pages 202 ff., on PCL see page 207, on PHB/PHV copolymers page 206; on polydioxanone PDS page 209. Discussion of a further bioresorbable material can for example be found in C A Bailey et al., J Hand Surg [Br] 2006 April;31(2):208-12.

Specific embodiments of non-degradable materials are: Polyetherketone (PEEK Optima, Grades 450 and 150, Invibio Ltd), Polyetherimide, Polyamide 12, Polyamide 11,Polyamide 6, Polyamide 66, Polycarbonate, Polymethylmethacrylate, Polyoxymethylene, or polycarbonateurethane (in particular Bionate® by DSM, especially Bionate 75D and Bionate 65D; according information is available on datasheets publicly accessible for example via www.matweb.com by Automation Creations, Inc.). An overview table of polymers and applications is listed in Wintermantel, page 150; specific examples can be found in Wintermantel page 161 ff. (PE, Hostalen Gur 812, Höchst A G), pages 164 ff. (PET) 169ff. (PA, namely PA 6 and PA 66), 171 ff. (PTFE), 173 ff. (PMMA), 180 (PUR, see table), 186 ff. (PEEK), 189 ff. (PSU), 191 ff (POM—Polyacetal, tradenames Delrin, Tenac, has also been used in endoprostheses by Protec).

The liquefiable material having thermoplastic properties may contain foreign phases or compounds serving further functions. In particular, the thermoplastic material may be strengthened by admixed fibers or whiskers (e.g. of calcium phosphate ceramics or glasses) and such represent a composite material. The thermoplastic material may further contain components which expand or dissolve (create pores) in situ (e.g. polyesters, polysaccharides, hydrogels, sodium phosphates), compounds which render the fusion device opaque and therewith visible for X-ray, or compounds to be released in situ and having a therapeutic effect, e.g. promotion of healing and regeneration (e.g. growth factors, antibiotics, inflammation inhibitors or buffers such as sodium phosphate or calcium carbonate against adverse effects of acidic decomposition). If the thermoplastic material is resorbable, release of such compounds is delayed. If the device is to be anchored not with the aid of vibration energy but with the aid of electromagnetic radiation, the liquefiable material having thermoplastic properties may locally contain compounds (particlulate or molecular) which are capable of absorbing such radiation of a specific frequency range (in particular of the visible or infrared frequency range), e.g. calcium phosphates, calcium carbonates, sodium phosphates, titanium oxide, mica, saturated fatty acids, polysaccharides, glucose or mixtures thereof.

Fillers used may include degradable, osseostimulative fillers to be used in degradable polymers, including: β-Tricalciumphosphate (TCP), Hydroxyapatite (HA, <90% crystallinity; or mixtures of TCP, HA, DHCP, Bioglasses (see Wintermantel). Osseo-integration stimulating fillers that are only partially or hardly degradable, for non degradable polymers include: Bioglasses, Hydroxyapatite (>90% cristallinity), HAPEX®, see S M Rea et al., J Mater Sci Mater Med. 2004 September; 15(9):997-1005; for hydroxyapatite see also L. Fang et al., Biomaterials 2006 July; 27(20):3701-7, M. Huang et al., J Mater Sci Mater Med 2003 July; 14(7):655-60, and W. Bonfield and E. Tanner, Materials World 1997 Janurary; 5 no. 1:18-20. Embodiments of bioactive fillers and their discussion can for example be found in X. Huang and X. Miao, J Biomater App. 2007 April ; 21(4):351-74), J A Juhasz et al. Biomaterials, 2004 March; 25(6):949-55. Particulate filler types include: coarse type: 5-20 μm (contents, preferentially 10-25% by volume), sub-micron (nanofillers as from precipitation, preferentially plate like aspect ratio >10, 10-50 nm, contents 0.5 to 5% by volume).

### BRIEF DESCRIPTION OF THE DRAWINGS

The invention is described in further detail in connection with the appended FIGS., wherein:

FIG. 1 shows a component of a so called bone anchored hearing aid as an example of a first type of the acoustic element according to the invention, the component being anchored in a cranial bone;

FIG. 2 shows a microphone of a hearing aid as an example of a second type of the acoustic element according to the invention, the microphone being anchored in bone of the outer auditory canal;

FIGS. 3 to 5 show exemplary embodiments of anchor elements suitable for the first type of acoustic element according to the invention;

FIGS. 6 to 8 show exemplary embodiments of anchor elements suitable for the second type of acoustic element according to the invention.

### DESCRIPTION OF THE PREFERRED EMBODIMENTS

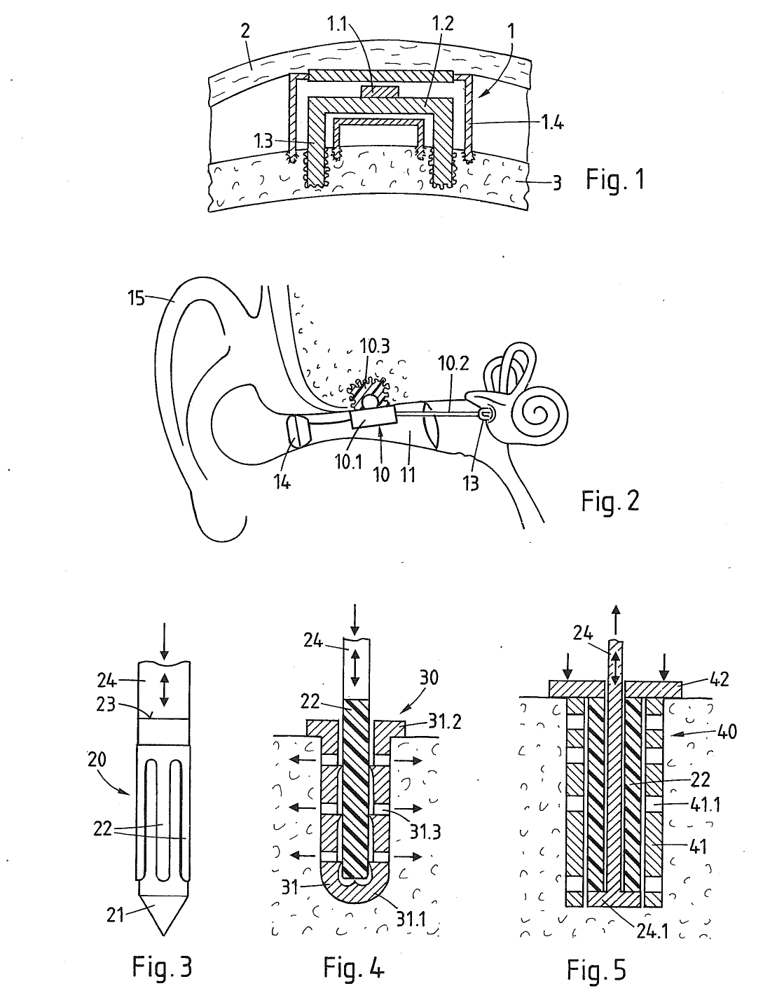

FIG. 1 shows an output transducer 1 of a so called bone anchored hearing aid, which is subcoutaneously (skin 2) implanted and anchored in a cranial bone 3 with the aid of anchor elements which are suitable for implantation with the method according to the invention, wherein the sonic vibration produced by the output transducer is to be coupled into the cranial bone as completely as possible. The output transducer comprises a transducer portion 1.1 (e.g. a piezoelectric element and an RF receiver for contact free reception of electric signals from a distanced microphone) and a mechanical resonator 1.2 (plate or beam) which is anchored in the bone tissue via anchor elements 1.3. The anchor elements 1.3 are rigidly fixed to the resonator 1.2. (transmitter cores of the anchor elements are e.g. integral parts of the resonator). The piezoelectric element is arranged to transmit sonic vibrations directly to the resonator 1.2. The arrangement of actuator 1.1, resonator 1.2 and anchor elements 1.3 is located in a housing 1.4, which is to prevent immobilization of the resonator 1.2 by tissue growth, wherein the four elements together constitute the acoustic element in the sense of the present description.

The resonator is e.g. a plate which is e.g. round with a plurality of anchor elements arranged around the plate periphery and the transducer portion 1.1 arranged in the plate center. In such an arrangement, a relatively small amount of input energy will result in relatively large resonator amplitudes. The resonator may also be a beam with the anchor elements arranged on the beam ends and the transducer portion arranged in the beam center. The resonator may have any suitable form.

For implantation of the acoustic element as illustrated in FIG. 1, the cranial bone 3 is exposed and openings for the anchor elements 1.3 are provided in the cranial bone, wherein the openings may be bores and preferably do not reach right through the cranial bone but have a blind end and wherein the housing 1.4 having an open top may be positioned first and used as a drill gauge. The assembly of resonator 1.2 and anchor elements 1.3 (possibly together with the open housing 1.4) is then positioned and the anchor elements 1.3 are anchored in the bone tissue by applying e.g. ultrasonic vibration to the anchor element locations of the resonator 1.2 or to the whole resonator 1.2. When all anchor elements 1.3 are anchored in the respective openings, the housing 1.4 is closed and the skin 2 sutured over the implanted acoustic element.

It is possible also to mount further components of the so called bone anchored hearing aid on the outer surface of the skin in the location where the resonator is subcoutaneously implanted, wherein such an external component may be kept in place by subcoutaneously implanted permanent magnets being anchored in the cranial bone and cooperating with magnets of the external component and wherein the external and implanted components are equipped for e.g. inductive transmittance of sound signals from the external component to the implanted component. Therein, the implanted permanent magnets are advantageously implanted with the same or a similar method as used for anchoring the anchor elements of the resonator.

In an analogue manner as described for the resonator assembly according to FIG. 1, it is possible also to anchor a transdermal post in a cranial bone and to couple a so called bone anchored hearing aid or a component (e.g. output transducer) of such a hearing aid to the anchored post. Coupling between the anchor element and other parts of the hearing aid or hearing aid component may be a per se known releasable coupling such as e.g. a snap connection, a threaded connection, or a bayonet catch.

Examples of anchor elements suitable for anchoring the acoustic element of FIG. 1 or other acoustic elements having similar functions in a cranial bone or other bone of the cranoimaxillofacial region are illustrated in FIGS. 3 to 5.

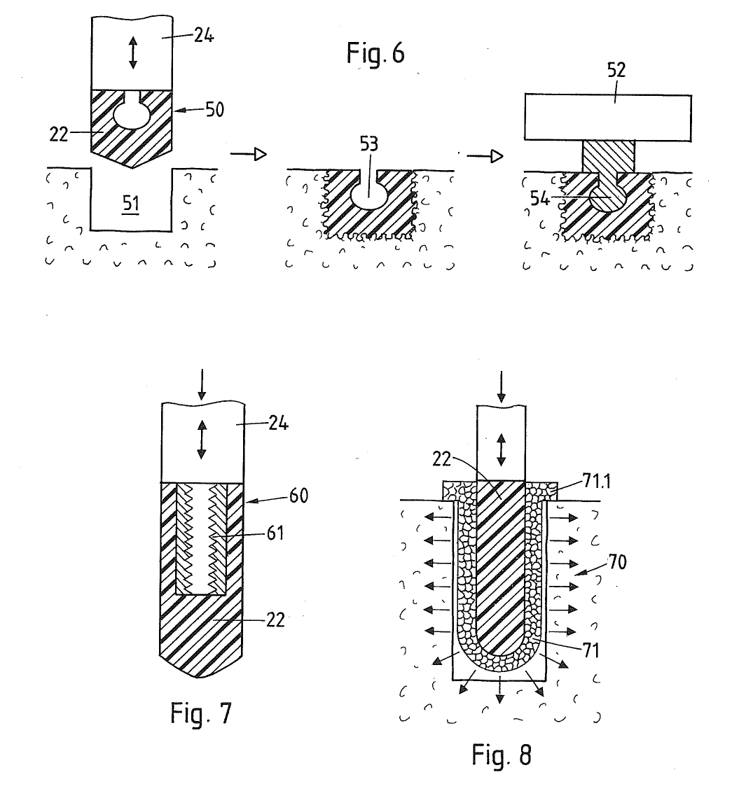

FIG. 2 illustrates an output transducer (acoustic element) of a hearing aid acting mechanically on the middle ear being implanted in the auditory canal 11 of a human ear with the method according to the invention. The output transducer comprises a transducer portion 10.1, a rigid transmitter beam 10.2 coupling the transducer portion to one of the auditory ossicles 13 of the middle ear and at least one anchor element 10.3. The transducer portion comprises e.g. a piezioelectric element being driven by an assembly 14 of a microphone (input actuator) and a sound processor which is e.g. positioned in the pinna 15. For preventing undesired disturbance of the transmitter beam function and therewith of the sound quality being transmitted to the auditory ossicle 13, it is important to sonically uncouple the output transducer 10 and in particular the transmitter beam 10.2 from the bone in which the output transducer is anchored. Therefore, anchor elements having an integrated damping function need to be applied. Examples of suitable such anchor elements are illustrated in FIGS. 6 to 8.

Similar arrangements can be made to directly couple the transducer to the inner ear, via a bore in the stapes (as is currently done for some middle ear implants), or to the base plate of the stapes (as long as the base plate itself is still flexible enough but the stapes as a whole is not suitable).

For implanting the output transducer according to FIG. 2, the bone of the auditory canal is exposed and at least one opening for the anchor element 10.3 is provided in the bone. The anchor element 10.3 is then implanted in the opening wherein for the implantation at least part of the material having thermoplastic properties and being comprised by the anchor element 10.3 is liquefied and made to penetrate into natural or specifically provided pores, cavities or other suitable structures of the bone tissue in the opening provided for the anchor element. After implantation of the anchor element(s) 10.3 the transducer portion 10.1 is coupled to the anchor element 10.3.

The coupling of the transmitter beam 10.2 to the auditory ossicle, such as the stapes, may be done in accordance with a method that is per se known, for example by a brace-like connection. In variants of this method, a polymer melt may be used to achieve an ideal positive fit connection. Such a polymer melt may for example be a melt of a bioactive, non-degradable polymer. The melt may, in an example, be created by the above-referenced technique involves by pushing an anchor element of liquefiable material between in a spacing while energy—for example ultrasonic vibrations—impinges on the anchor element to liquefy it.

In other embodiments, the coupling between the transmitter beam 10.2 and the auditory ossicle (or ossicle part or inner ear) can, in addition or as an alternative to the described anchoring of an anchor element 10.3, be achieved by the herein described anchoring technique that involves liquefaction of a liquefiable element by impinging energy and making the liquefied material penetrate into cavities, pores or other suitable structures of the tissue and by letting the liquefied material re-solidify to form a positive fit connection.

In this, an anchoring element for this anchoring technique may be of a kind described hereinafter referring to the following figures. Alternatively, an anchoring element may be brace-like and with a bendable core not liquefiable under implantation conditions and a thermoplastic portion that may for example be a full or partial (e.g. interior) coating of the brace-like core. The anchoring will then comprise a first anchoring sub-step that comprises mechanically bending the anchoring element and a second anchoring sub-step that comprises coupling energy into the anchoring element to at least partially liquefy the coating to achieve a micro-form-fit connection. FIGS. 3 to 5 illustrate three exemplary embodiments of anchor elements suitable for anchoring an acoustic element in a bone of the craniomaxillofacial region, wherein the anchor element is to function as a sound transmitter between other parts of the acoustic element attached or attachable to the anchor element, and the bone tissue in which the anchor element is anchored, and for this purpose comprises a transmitter core of a material which is able to transmit sound with as little loss as possible.

The anchor element 20 according to FIG. 3 comprises a transmitter core 21 whose outer surface is partly covered with the material 22 having thermoplastic properties. The anchor element 20 has a proximal face 23 suitable for coupling energy into the anchor element, e.g. ultrasonic vibration energy coupled into the anchor element by applying a sonotrode 24 of an ultrasonic device (not shown) to the proximal face 21. The anchor element 20 further comprises surfaces to be brought into intimate contact with the bone tissue in the opening provided for anchoring the anchor element, wherein these surfaces comprise surface regions of the transmitter core on the one hand and surface regions consisting of the material 22 having thermoplastic properties and covering core surfaces on the other hand. The surfaces of the transmitter core 21 not being covered with the material 22 are located e.g. at a more or less sharp distal end of the anchor element and/or in concave regions of a circumferential surface or the anchor element. Advantageously, the material 22 having thermoplastic properties fills recesses in the transmitter core such that the two kinds of surfaces are flush with each other or such that energy directors (ridges or humps provided on the surfaces of the material 22) protrude over the flush surfaces.

For implantation of the anchor element 20 according to FIG. 3, an opening is provided in bone tissue, the opening having a cross section which corresponds with the cross section of the anchor element 20 such that on insertion of the anchor element into the opening uncovered circumferential surfaces of the transmitter core get into intimate contact with the bone tissue inside the opening, and, if necessary for the in situ liquefaction of the material 22, such that there is friction between the material 22 and the bone tissue. If, as illustrated in FIG. 3, the transmitter core 21 of the anchor element 20 has an uncovered, more or less sharp distal end, the depth of the opening provided for the anchor element is smaller than the axial length of the anchor element such that on implantation of the anchor element this distal end is dug into the bottom of the opening and therewith gets into intimate contact with the bone tissue.

The anchoring element 30 illustrated in FIG. 4 comprises a transmitter core 31 which has the form of a hollow and perforated or fenestrated sleeve and the material 22 having thermoplastic properties is arranged or arrangeable inside the core 31. The sleeve has a closed distal end 31.1 and possibly a proximal flange 31.2 and its inner surface is preferably equipped with protruding edges or points which act as energy directors.

For implantation of the anchoring element 30, an opening is provided in the bone tissue, wherein the opening has a cross section and preferably a depth also which are adapted to the transmitter core 31 such that on introduction of this core into the opening, as much as possible of the outer core surface is in intimate contact with the bone tissue inside the opening. The material 22 introduced in the transmitter core 31 is liquefied at least partly, advantageously by ultrasonic vibrational energy which is coupled into the material 22 by applying the sonotrode 24 to its proximal side and to press it into the core 31, wherein the pressing force is counteracted by the bone surface supporting the proximal flange 31.2 of the core or by the bone tissue at the bottom of the opening. The liquefied material 22 is pressed through the perforations or fenestrations 31. 3 of the core to penetrate the bone tissue, wherein the tight fit of the core in the opening and possibly a distal core end without perforation or fenestration prevents the liquefied material from spreading over the whole outer surface of the core 31.

Anchor elements similar to the ones illustrated in FIGS. 3 and 4 as well as further embodiments of such anchor elements are disclosed in the publication U.S. Pat. No. 7,008,226 whose disclosure is enclosed herein by reference in its entirety.

The anchor element 40 according to FIG. 5 again comprises a transmitter core 41 which is hollow and comprises perforations or fenestrations 41.1 and a distal end which may be closed or open. The material 22 has the form of a tube adapted in cross section to the space inside the transmitter core 41. The sonotrode 24 extends through the tube and the tube is kept on the sonotrode by a foot piece 24.1 forming the distal end of the sonotrode 24.

For implantation of the anchor element 40 according to FIG. 5, an opening is provided in the bone tissue, the cross section of the opening and possibly its depth being adapted to the cross section and possibly the axial length of the transmitter core 41 such that the circumferential surface of the core 41 and possibly also its distal face can be brought into intimate contact with the bone tissue in the opening on introduction of the core 41 into the opening. The tube of the material 22 arranged on the sonotrode 24 is introduced into the transmitter core preferably with the interface between the foot piece 24.1 and the distal face of the tube being located in the vicinity of the deepest perforation or fenestration 41.1. The sonotrode is activated and pulled in a direction out of the core 41 while the tube of the material 22 is held against the foot piece 24.1 with the aid of a counter element 42. The material 22 having thermoplastic properties is liquefied at the interface between the foot piece 24.1 and the distal face of the tube and flows through the core perforations or fenestrations 41.1 to penetrate the bone tissue surrounding the core as above described in connection with FIG. 4.

Anchoring elements which are similar to the one illustrated in FIG. 5 and the corresponding anchoring method are disclosed in further detail in the application PCT/CH2009/000138 (not published yet), the content of which is enclosed herein in its entirety by reference.

FIGS. 6 to 8 illustrate three exemplary embodiments of anchor elements suitable for anchoring an acoustic element in a bone of the craniomaxillofacial region, wherein the anchor element has an integrated damping function which prevents sound transmission between the acoustic element and the bone in which it is anchored. For this purpose the anchor element consists fully of the material 22 having thermoplastic properties or this material forms an interface between a core which in itself may have transmitter properties and the bone tissue.

The anchor element 50 according to FIG. 6 consists fully of the material 22 and is anchored in an opening 51 e.g. with the aid of a sonotrode 24 which is applied to its proximal face. FIG. 6 shows the anchor element 50 before implantation (left), implanted (middle) and with a further part 52 of the acoustic element attached thereto. For such attachment the anchor element 40 may comprise attachment means in the region of its proximal end, such as e.g. an undercut opening 53 into which a corresponding protrusion 54 of the further part is snapped. Instead of comprising an attachment means the proximal face of the anchor element 50 may be even and the further part 52 may be equipped with a thorn or thorns, witch may be equipped with barbs and for coupling the further part 52 to the anchor element 50 is forced into the implanted anchor element 50 e.g. with the aid of ultrasonic vibration. Such coupling is advantageous as positioning of the anchor element in the bone does not need to correspond exactly with the desired position of the further part 52 and/or only one anchor element 50 with a correspondingly larger proximal face may receive a plurality of thorns of the further part 52.

The anchor element 60 according to FIG. 7 is similar to the anchor element of FIG. 6 but further comprises a core which may in itself be capable of transmitting sound but which is prevented from transmitting sound between the acoustic element of which it is part and the bone tissue in which it is anchored by its surfaces being fully covered with the material 22 where it is to be located in the opening provided for the implantation. The core may have the function of strengthening an attachment means, e.g. in the form of an inner thread which is to cooperate with a threaded bolt attached to a further part of the acoustic element to be coupled with the anchor element 60.

The anchor element 70 according to FIG. 8 comprises a core 71 in the form of a sleeve consisting of a material having an open porosity and the material 22 is arranged inside the sleeve. The sleeve has a closed distal end and preferably a proximal flange 71.1 to be supported on the bone surface around the opening which is provided in the bone for implanting the anchor element 70.

For implantation of the anchor element 70 an opening is provided in the bone tissue, wherein the opening is dimensioned (regarding diameter and depth) such that the core 71 is able to loosely fit into it. The material 22 is arranged in the core 71, liquefied at least partly by application of e.g. vibrational energy and pressed through the core wall to fill the space between wall and bottom of the opening and to penetrate the bone tissue of this wall and bottom and therewith constitute not only a positive fit connection between the anchor element 70 and the bone tissue but also a damping interface.

If by chance, not the whole outer surface of core 71 is covered by the liquefied material, uncovered core surface regions will be distanced from the bone tissue and therefore will not be able to form sound conducting bridges between the core and the bone tissue.

Similar anchor elements as illustrated in FIGS. 6 to 8 and methods for implanting such anchor elements are disclosed in the publication U.S. Pat. No. 7,335,205, the disclosure of which is enclosed herein in its entirety by reference.

1. A method for implanting an acoustic element in a bone of the craniomaxillofacial region, the method comprising the steps of:
providing an acoustic element with at least one anchor element, the anchor element being an integral part of the acoustic element or a separate item, wherein the anchor element comprises a material having thermoplastic properties,
providing an opening in the bone for implantation of the at least one anchor element,
positioning the anchor element in the opening, and
anchoring the anchor element in the opening by applying energy to the anchor element for a time sufficient for liquefying at least part of the material having thermoplastic properties and making it penetrate into cavities, pores or other suitable structures of the bone tissue in the opening and by letting the liquefied material re-solidify to form a positive fit connection between the anchor element and the bone tissue, where it has penetrated the cavities, pores or other suitable structures of the bone tissue.

2. The method according to claim 1, wherein the anchor element is a separate item and the method further comprises a step of attaching further parts of the acoustic element to the anchor element, the step of attaching being carried out before the step of positioning or after the step of anchoring or simultaneously with the step of anchoring.

3. The method according to claim 1, wherein, for achieving a connection between the acoustic element and the bone tissue which is capable of transmitting sound from the acoustic element to the bone tissue, the anchor element comprises a transmitter core of a material capable of sound transmission and comprising surfaces which at least partly are equipped for furthering osseointegration, wherein the liquefiable material is arranged to partly cover surfaces of the transmitter core or wherein the transmitter core is a perforated or fenestrated sleeve and the liquefiable material is arranged or arrangeable on the inside of the core, and wherein the opening provided for anchorage of the anchor element is dimensioned such that on positioning, core surfaces not covered with the material having thermoplastic properties can be brought into intimate contact with the bone tissue in the opening.

4. The method according to claim 3, wherein the bone of the craniomaxillofacial region is a cranial bone and the acoustic element is a so called bone anchored hearing aid or a component of such a hearing aid.

5. The method according to claim 1, wherein, for achieving a connection between the acoustic element and the bone tissue which does not transmit sound between the acoustic element and the bone tissue, the anchor element consists fully of the material having thermoplastic properties, comprises a core which as far as to be introduced in the opening is covered fully with the material having thermoplastic properties.

6. The method according to claim 1, wherein, for achieving a connection between the acoustic element and the bone tissue which does not transmit sound between the acoustic element and the bone tissue, the anchor element comprises a core in the form of a sleeve of a open-porous material in which sleeve the material having thermoplastic properties is arranged or arrangeable, and wherein the opening provided for anchoring of the anchor element is dimensioned for the core to sit loosely therein.

7. The method according to claim 5, wherein the bone of the craniomaxillofacial region is an otic capsule bone, a mastoid bone or a cranial bone and the acoustic element is a component of a hearing aid acting mechanically or electrically on the ear.

8. An acoustic element comprising:
at least one anchor element,
the anchor element being an integral part of the acoustic element or a separate item,
wherein the anchor element comprises a material having thermoplastic properties and wherein the acoustic element and/or the anchor element are equipped for transmitting energy to the material having thermoplastic properties.

9. The acoustic element according to claim 8, wherein the at least one anchor element comprises a transmitter core of a material suitable for transmitting sound and having surfaces which are at least partly equipped for furthering osseointegration and wherein the material having thermoplastic properties coats part of the outer surfaces of the core or the core is perforated and the material having thermoplastic properties is arranged or arrangeable within the core.

10. The acoustic element according to claim 9, wherein the core of the anchor element is rigidly connected or connectable with the acoustic element.

11. The acoustic element according to claim 9, constituting a component of a so called bone anchored hearing aid.

12. The acoustic element according to claim 8, further comprising a mechanical resonator and a transducer portion arranged for exciting the mechanical resonator.

13. The acoustic element according to claim 8, wherein the at least one anchor element fully consists of the material having thermoplastic properties or comprises a core which at least as far as it is to be introduced in the opening is fully covered by the material having thermoplastic properties.

14. The acoustic element according to claim 13, constituting a component of a hearing aid acting mechanically or electrically on the ear.
